(19) United States
(12) Patent Application Publication
(10) Pub. No.: US 2013/0304080 A1
(43) Pub. Date: Nov. 14, 2013

(54) Cesarean Delivery Incision Template

(71) Applicant: Thomas Barry Landry, Boulder, CO (US)

(72) Inventor: Thomas Barry Landry, Boulder, CO (US)

(21) Appl. No.: 13/470,258

(22) Filed: May 12, 2012

(51) Int. Cl. A61B 17/42

(52) U.S. Cl. 606/121

(57) ABSTRACT

Cesarean deliveries account for greater than 30% of the deliveries in the United States. The Cesarean Delivery Incision Template is a tool which assists the surgeon in making the incision uniform in the vertical as well as horizontal plane.

The Cesarean Delivery Incision Template is made of a transparent pliable material that easily conforms to the patient's body. Once in place, the surgeon can either use a marking pen to draw a line where the incision it to be placed or run a scalpel along the template to make the incision. The initial incision will be used in all subsequent cesarean deliveries on the patient making the initial placement very important.

### CROSS REFERENCE TO RELATED APPLICATIONS

Not applicable.

### BACKGROUND

1. Field of the Invention

This invention relates to apparatus and methods for surgical procedures. With more particularity, this invention relates to a guide or template for use by a physician to properly locate an incision on a patient's abdomen for a cesarean delivery.

2. Related Art

For various reasons well known in the medical community, many babies are delivered via Cesarean delivery. This invention is directed toward assisting the surgeon in making the incision on the mother's abdomen, fundamental for a Cesarean delivery. Typically, in preparation for such a delivery, the patient is placed in a supine position, tilted slightly to one side (for example, to the patient's left). It is important to make the incision in the proper location on the patient's body. The typical placement of the patient as described, combined with the distortion of the patient's anatomy as a result of the pregnancy, can make it difficult for the surgeon to make the incision in the proper location and orientation, as described further below.

It is helpful to visualize an imaginary line down the center of the patient's entire body (including the abdomen), which is well known in the medical field as the line of bilateral symmetry. The line of bilateral symmetry can be described as follows: with the patient standing vertically, the line of bilateral symmetry is an imaginary vertical line (in the direction of the patient's height aspect), centered in a horizontal aspect (that is, side to side), and essentially bisecting the patient's body. FIG. 1/6 illustrated the location of the line of bilateral symmetry, denoted as LS in the drawing.

The Cesarean incision, at the point where the incision crosses said line of symmetry, is transverse to the line. The incision must be made in the proper location along the length of the patient's body; and in addition should be centered on the line of bilateral symmetry so that each half of the incision is symmetric thereabout. This means that there is equal incision length on either side of the line of bilateral symmetry, and that the arc of the incision is level on both sides of the midline.

The first Cesarean delivery incision made on the patient is frequently referred to as the “primary” incision. Since any future Cesarean delivery incisions are generally made along the very same line, with the primary incision acting as a guide for any subsequent incisions, it is especially important that the primary incision is equally distributed across the midline of the body (LS). It is also important that the angle of the arc of the incision be mirror images on either side of the midline.

Presently, surgeons use sterile marking pens to draw the Cesarean delivery incision line free hand on the patient's abdomen. While this technique may provide some advantage over no marking at all, it can be appreciated that any free hand technique may lack the desired accuracy for placement of the incision.

The present invention comprises apparatus and method which assist the surgeon in properly locating Cesarean delivery incisions. It can also provide a template for incisions made in the same area for surgeries other than cesarean sections. This will provide a more desirable cosmetic outcome as well as a permanent template for future incisions in this area of the body.

### BRIEF DESCRIPTION OF THE DRAWINGS

FIG. 1/6 is a schematic of the human body, illustrating the line of bilateral symmetry of the body (LS).

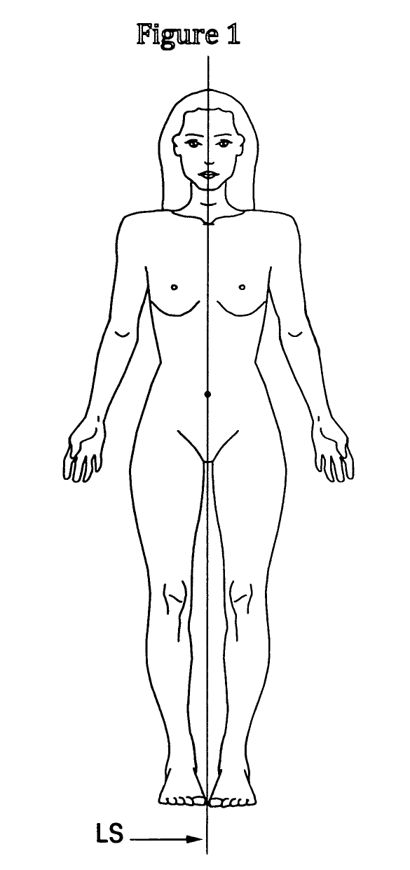

FIG. 2/6 is a view of the template in a shape generally conformed to the patient's body, viewed from one end.

FIG. 3/6 is one view of one embodiment of the template of the present invention, generally with the apparatus flattened out and viewed from above.

FIG. 4/6 is a perspective view of the template. The view is what the patient would see if she were looking down at the template while placed on her body.

FIG. 5/6 is a side view of the template.

FIG. 6/6 is a view of the template in place on a patient's body, with the surgeon in the process of marking the line where the Cesarean delivery incision will be made on the patient's body.

### DESCRIPTION OF THE PRESENTLY PREFERRED EMBODIMENT(S)

Various embodiments of the present invention may be made without departing from the scope of the invention. By way of illustration, one of the presently preferred embodiments will be described, with reference to the drawings.

Generally, the apparatus of the present invention comprises a template which is placed against the patient's body and is used as a guide for marking a patient's body for a Cesarean delivery incision. With more particularity, in a broad embodiment, the present invention comprises a template which aligns with the body's natural line of bilateral symmetry, and provides an edge or surface transverse to the line of bilateral symmetry, at the point crossing the same. The upper template edge or surface is marked as to distance on either side of the midline of the template, aligned with the line of bilateral symmetry. An incisions line may be marked along the edge or surface so as to be symmetric about the line of bilateral symmetry.

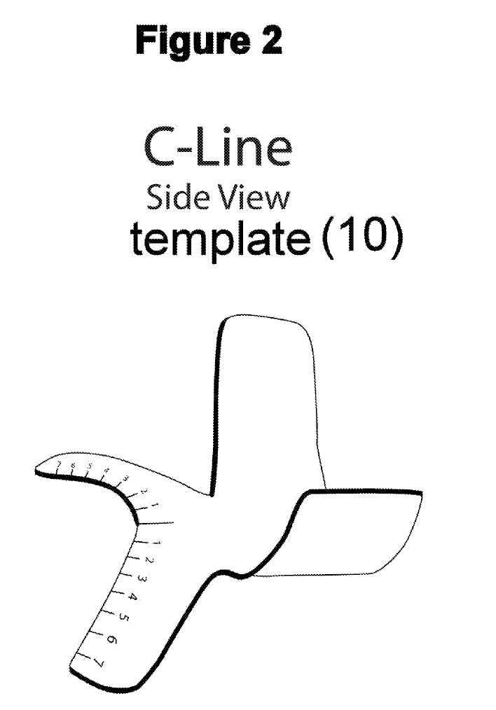

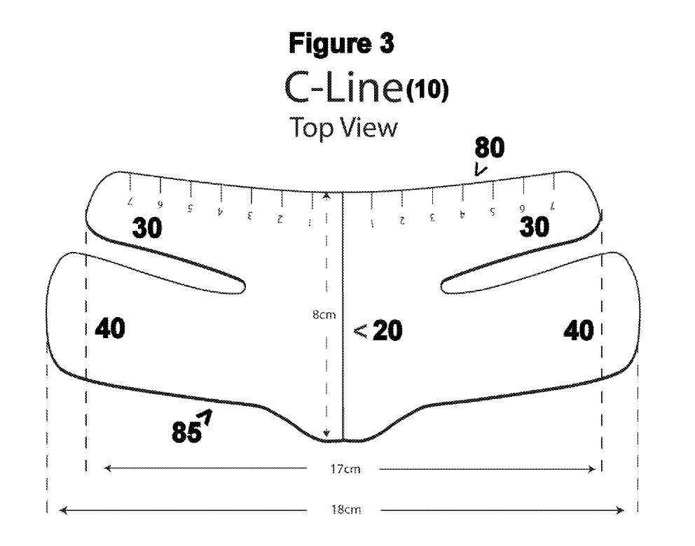

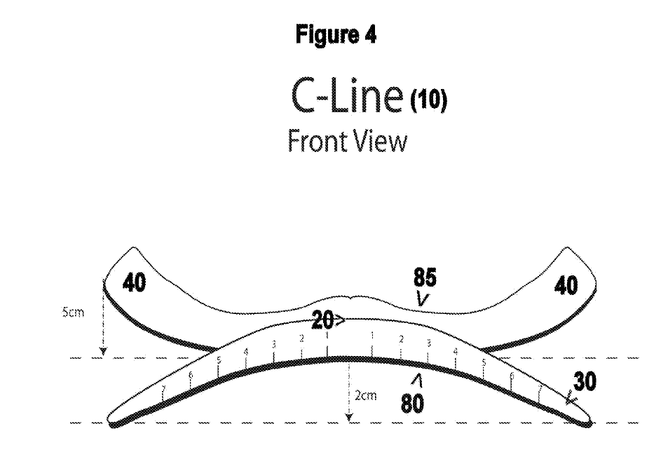

In the presently preferred embodiment, as best seen in FIG. 2/6, the template (10) is made from a thin, flexible sheet material, such as plastic, which can be sterilized and conformed to the shape of the patient's body (namely, the abdomen) when placed against the same. FIG. 3/6 is a view of the template (10), generally flattened out and viewed from above. Template (10) is symmetric about a midline (20). Template (10) bears indicia of midline (20), such as a colored line, a groove, or a raised line. Template (10) comprises guide section (30) extending to either side of midline (20), with an upper template edge (80), along which the incision line is marked, as will be later described. Preferably, template (10) also comprises of handles (40), extending to each side of midline (20). Both guide section (30) and handles (40) assist the surgeon and the surgeon's assistant to hold the template (10) in place. As will be described in more detail below, and as can be seen in FIGS. 4/6 and 5/6, handles (40) are preferable shaped so as to be generally conformable to the uppermost portion of the patient's leg and crotch area (namely, that portion of the patient's body where the legs join the torso). The symmetric aspect of this portion of the human anatomy, combined with the conformed shape of the handle (40), assist in assuring that the template (10), and more particularly midline (20), are aligned with the line of bilateral symmetry.

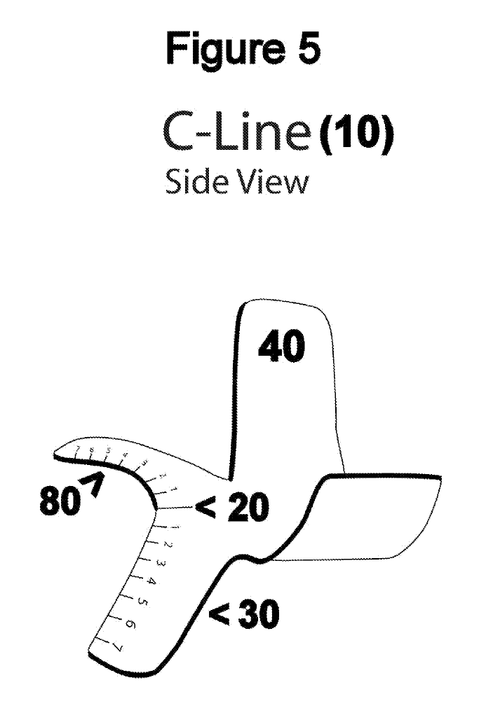

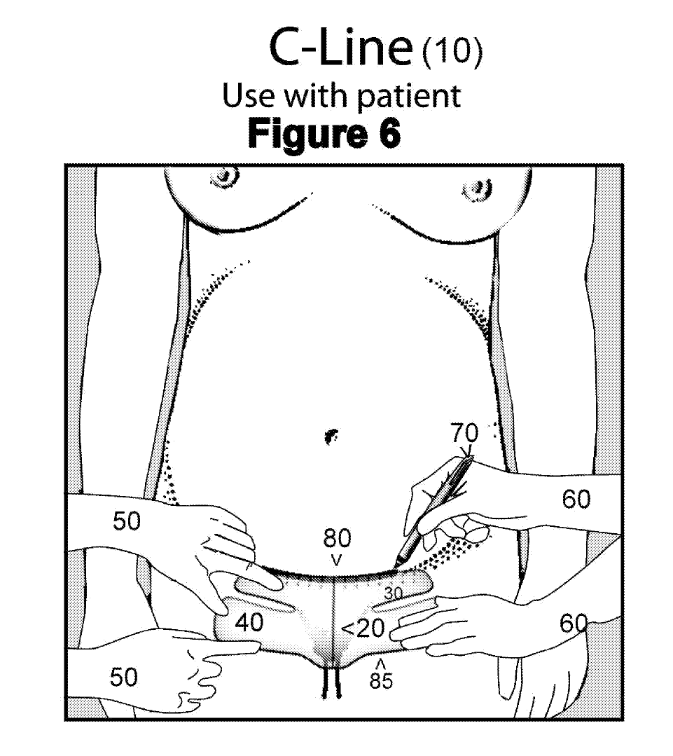

As can be seen in FIG. 6/6, when midline (20) and handles (40) are properly aligned on the patient's body, guide section (30) is necessarily aligned. Template (10) is preferably made with a single piece of material. Guide section (30), as can be seen in the drawings, especially FIG. 5/6, is preferably shaped to yield an upper template edge (80) generally conforming to the patient's abdomen, usually in a shallow curve or arc. Preferably, guide section (30) comprises markings spaced at equal distances on either side of midline (20), for example, marking to 4 on one side of midline (20) to 4 on the opposite side of midline (20). The markings will be spaced on either side at unit distances (e.g. centimeters) on either side of the midline (20).

As described above, template (10) is preferably made from a flexible, relatively thin sheet material, such as plastic. Preferably, the entirety, or at least the portion along midline (20) of template (10), is made of a material which is sufficiently transparent or see-through that anatomical features of the patient can be seen through template (10). Material such as thin plastics can be sterilized, and can be molded so as to assume a desired shape in a relaxed position, yet can be conformed within reasonable limits to other desired shapes. FIG. 4/5 is a view of template (10), generally in a shape conforming to a patient's body, viewed substantially from one end; this is the view which a patient would most nearly see if she looked down at template (10) with template (10) in place on her body. FIG. 5/6 is a perspective view of template (10), showing guide section (30) and handles (40) in their relaxed position (conforming generally to a patient's body). Preferably, template (10) comprises a lower edge (85) which generally conforms to the shape of the junction of a patient's leg with the torso, as can be readily seen in the drawing.

Dimensions of template (10) may be varied to suit the user. By way of example only, template (10) may measure approximately 8 centimeters along midline (80), have a width of guide section (30) of approximately 17 centimeters, and a width of handles (40) of approximately 18 centimeters. Larger or smaller templates (10) can be used to accommodate larger or smaller patients.

As described in the preceding write-up and the drawings, template (10) is advantageously formed so as to assume the approximate position shown in FIG. 4/6 in its “natural” or unstressed position. Such position conforms reasonably closely to the average patient's body, therefore, easing the placement of template (10) in place while marking the line for the incision as in FIG. 6/6.

### Use of the Template of the Present Invention:

FIG. 6/6 shows template (10) in use. Fundamentally, template (10) must be positioned so that midline (20) lies along the body's line of symmetry, with guide section (30), and more particularly upper template edge (80), at the suitable portion along the length of the patient's body. In practice, there are several attributes of the human body which assist in placement of the midline of the template (10) along the line of bilateral symmetry. As previously noted, preferably template (10) is made of a clear or transparent material, or with a clear center, so that it is sufficiently “see through” that the position of midline (20) on the body can be accurately noted. In pregnant women, a line known as the linea alba becomes more apparent due to hyper pigmentation. It can be used as a reference for the placement of midline (20). In addition, the navel, pubic symphysis, and the convergence of the labia (both of which are at the junction of the patient's legs), are also anatomically mid-line, or along the bilateral line of symmetry (LS). The surgeon can use these points to properly align template (10), by orienting midline (20) along an imaginary line joining them. It is further noted that in the preferred embodiment shown, handles (40) generally conform to the contour of the patient's legs, naturally centering template (10).

With midline (20) of template (10) properly oriented along the line of bilateral symmetry, template (10) must be positioned at an appropriate distance along the patient's length, so that upper template edge (80) is at a proper vertical location. This vertical placement (“inferior” or “superior”), as same would be in the relevant art is a matter of judgment by the surgeon. By way of example only, a common vertical spacing is to make the incision approximately the width of 2 fingers (index and middle fingers) above the top or superior edge of the pubic bone.

Referring to FIG. 6/6, with template (10) generally placed as is in the preceding description; an exemplary use can be described. Template (10) is held against the patient's body, positioned so that handles (40) and lower template edge (85) are conformed substantially to the upper leg and crotch area of the body (where the patient's legs join the torso). This naturally centers template (10) on the bilateral line of symmetry of the patient. In addition, midline (20) is visually aligned along the several natural markers of the line of bilateral symmetry as described above (linea alba, navel, pubic symphysis, and convergence of the labis). Template (10) is sufficiently transparent that when placed against the patient's skin, attributes such as the linea alba, area of the pubic symphysis and the convergence of the labia can be seen through template (10). Preferably, template (10) is held in place by a surgical assistant (whose arms/hands are designated as 50), and by the surgeon (whose arms/hands are designated as 60). The surgeon then uses a sterile marker (70) to mark the patient's body for the cesarean delivery incision, using upper template edge (80) of the guide section (30) of template (10). Thereafter, template (10) is removed from the patient's body, and the incision is made along the line so marked. It is to be understood that if desired, the surgeon could make the incision without pre-marking; that is, by running a scalpel along the upper template edge (80) with template (10) in place.

### CONCLUSION

While the preceding description contains much specificity, it is to be understood that same are presented only to describe some of the presently preferred embodiments of the invention, without departing from the scope thereof.

For example, dimensions of the template (10) can be changed to suit the user; particular shape and contour of guide section (30), or more particularly upper template edge (80), can be varied from a generally curved shape to a generally straight line shape (with little or no curvature); different material can be used, such as various types of thin, flexible plastic or similar materials; the shape of the handles (40) and guide section (30) can be modified as desired; and if desired, handles (40) can be omitted.

Therefore, the scope of the invention is to be determined not but the illustrative examples set forth above, but by the appended claims and their legal equivalents.

I claim:

1: A template for guiding the placement of an incision for a Cesarean delivery or other lower abdominal surgeries, comprising: a flexible sheet comprising indicia permitting a midline of said flexible sheet to be aligned with a line of bilateral symmetry of the human anatomy at a desired location, further comprising a guide section with the upper template edge which crosses said line of bilateral symmetry at a right angle, and wherein said guide section can be conformed to the contour of the abdomen of the pregnant human body.

2: The template of claim 1, whereby said flexible sheet comprises markings proximal to said upper template edge spaced at uniform distances on either side of said midline.

3: The template of claim 2, wherein said flexible sheet is sufficiently transparent that, when placed against a human, characteristics of the anatomy of said human can be viewed through.

4: The template of claim 3, further comprising handles extending outwardly from said midline, and wherein a lower template edge is shaped to conform to the shape of the pregnant human body where the legs of said human body where the legs of said human body join the torso of said human body.

5: The template of claim 4, wherein said template is made of a plastic material.

6: A template for placement of a primary Cesarean delivery incision on a human female, comprising: a flexible sheet having a midline marked thereon and being symmetric about said midline, an upper template edge transverse to said midline and comprising markings placed at uniform distances on either side of said midline, a handle extending outwardly from said midline, and a lower template edge having the shape which conforms to the shape of the crotch area of a human body, and wherein said sheet is conformable to the body of a pregnant human female.

7: The template of claim 6, wherein said flexible sheet is sufficiently transparent that, when placed against a human, characteristics of anatomy of said human can be viewed there through.

8: A method of making a primary Cesarean delivery incision on a human body, comprising of the steps of:
Placing a template on said human body, said template having indicia thereon which align said template with the body's line of bilateral symmetry, said template comprising and edge which conforms to the shape of said human body and transversely crosses said line of bilateral symmetry at a desired location along the length of said human body; and
Marking a line on the skin of said human body along said edge and of equal length on either side of said line of bilateral symmetry.

9: The method of claim 8, further comprising the stop of making an incision along said line.
